(19) United States
(12) Patent Application Publication
(10) Pub. No.: US 2024/0082493 A1
(43) Pub. Date: Mar. 14, 2024

(54) FLUID DELIVERY DEVICES AND METHODS THEREOF

(71) Applicant: TAKEDA PHARMACEUTICAL COMPANY LIMITED, Osaka (JP)

(72) Inventors: Shekhar NIMKAR, Swampscott, MA (US); Arnaz MALHI, Lexington, MA (US); William G. ATTERBURY, Columbus, OH (US); Jessica Diane YOUNG, Columbus, OH (US); Beverly Anne PIATT, Columbus, OH (US); James Alan PRESCOTT, Columbus, OH (US); Adam C. FOX, Columbus, OH (US)

(73) Assignee: TAKEDA PHARMACEUTICAL COMPANY LIMITED, Osaka (JP)

(21) Appl. No.: 18/037,834

(22) PCT Filed: Nov. 22, 2021
(86) PCT No.: PCT/JP2021/044376

§ 371 Date: May 19, 2023

Related U.S. Application Data
(60) Provisional application No. 63/117,538, filed on Nov. 24, 2020.

(51) Int. Cl. A61M 5/24; A61M 5/31; A61M 5/315

(52) U.S. Cl. CPC A61M 5/2448; A61M 5/3146; A61M 5/31551; A61M 2005/3132; A61M 2005/2407

(57) ABSTRACT

Fluid delivery devices for delivering therapeutic agents are described. More particularly, this disclosure relates to drug reconstitution devices having a plurality of components movable with respect to one another such that an active agent and a solvent can be reconstituted within the device prior to injection. The fluid delivery devices can include a housing, a cartridge holder, a cartridge disposed within the cartridge holder, a button rod, and a foot. The cartridge stores the active agent and the solvent, which are separated by one or more stoppers. The button rod and foot can provide an axial force upon the stopper(s) in the cartridge to inject the fluid once reconstituted. The foot and the button rod can be separate components, thereby enabling the foot to rotate independently with respect to the button rod as the cartridge holder is rotated to reconstitute the therapeutic and prime the device for injection.

### FIELD OF THE DISCLOSURE

This disclosure generally relates to fluid delivery devices for delivering therapeutic agents. More particularly, this disclosure relates to drug reconstitution devices having a plurality of components movable with respect to one another such that an active agent and a solvent can be reconstituted within the device prior to injection.

### BACKGROUND ART

Reconstitution is the process of mixing a lyophilized active agent with a solvent so the active agent can be re-liquefied, or reconstituted, before injection. The process is common, since lyophilizing (or freeze-drying) an active agent, such as a drug, biologic, or other therapeutic, can help to preserve the shelf life of the agent and enable the agent to be delivered to the end user for future mixing. That said, many times the mixing of the agent is expected to be completed by someone other than the manufacturer or a pharmacist, so care must be taken to ensure that the mixing of the solvent and the active agent is accurate and error-proof.

Prior designs for reconstitution devices included a multi-stopper cartridge that housed the solvent between two stoppers and the active agent between the distal of the stoppers and the end of the cartridge. In these designs, the proximal stopper moves axially, pushing the solvent through the distal stopper such that it flows into the space of the cartridge storing the active agent. Once the solvent interacted with the lyophilized agent, the cartridge can be shaken to reconstitute the therapeutic agent. Although prior designs provide a technique to ensure accurate amounts of solvent and active agent were provided in the same cartridge, they do not provide a multitude of redundancies and safety protocols to ensure (1) they were properly mixed and (2) they were properly administered to the patient after mixing. There is therefore a need for improved methods, devices, and systems that accurately reconstitute lyophilized active agents while also providing an error-preventing, integrated apparatus that can be used to administer the reconstituted agent.

### SUMMARY OF THE INVENTION

It is an object of the present disclosure to provide systems, devices, and methods to reconstitute an active agent while also providing redundancies and feedback to ensure accurate dosing.

The present disclosure provides a fluid delivery device. The fluid delivery device can include a housing including an internal thread. The fluid delivery device can include a cartridge holder mechanically coupled to the housing. The cartridge holder can include an external thread extending at least partially along a length of the cartridge holder. At least a portion of the external thread can wrap 360° around the cartridge holder. The fluid delivery device can include a cartridge disposed within a cartridge holder internal cavity. The fluid deliver device can include a button rod. The button rod can include a retaining hook, a demounting stop extending at a non-zero angle from a longitudinal axis of the button rod, a holding snapper, a bypass snapper extending at a non-zero angle from the longitudinal axis of the button rod, and a drive button.

The housing can include a button rod groove, and the button rod can include a wing sized to engage the button rod groove. A first length of the button rod between the wing and a distal end of the button rod (e.g., a dosage length) can correspond to a volume of fluid expelled from the fluid delivery device.

The fluid delivery device can include a foot coupled to a distal end of the button rod. The foot can include a pusher to push against stoppers in the cartridge. The foot can include a plurality of flexible fingers radially arranged around a common axis extending along the length of the foot.

The cartridge holder can have an extended configuration and a retracted configuration with respect to the housing. In the extended configuration, each flexible finger of the plurality of flexible fingers can engage a proximal end of the cartridge. In the retracted configuration, each flexible finger of the plurality of flexible fingers can engage an internal surface of the cartridge. In the retracted configuration, the demounting stop can abut a housing stop positioned proximate a proximal end of the housing.

The foot can include a foot groove sized to engage a foot snap of the button rod. The foot can be detachably attachable to the distal end of the button rod.

The cartridge holder can be rotatable with respect to the housing. The retaining hook can be inwardly deflectable by the proximal end of the cartridge holder. The housing can include a holding snapper aperture sized to engage the holding snapper.

The internal thread can wrap at least 360° around an interior surface of the housing. The internal thread can wrap at least 420° around an interior surface of the housing. The internal thread can include a beveled first end and a beveled second end to reduce friction as the cartridge holder rotates with respect to the housing.

The housing can include an axial stop sized to engage a proximal end of the cartridge holder. The cartridge holder can have an extended configuration and a retracted configuration with respect to the housing. The axial stop can be positioned to inhibit the cartridge holder from sliding proximally beyond a predetermined position, thereby reducing the chance or likelihood of underdosing.

The cartridge can include a distal tip, a first stopper, a second stopper disposed between the distal tip and the first stopper, an active agent disposed between the distal tip and the second stopper, and a solvent disposed between the second stopper and the first stopper. The fluid delivery device can include a foot coupled to a distal end of the button rod and including a pusher and a plurality of flexible fingers extending radially from the foot and centering the pusher centrally with respect to the first stopper. The fingers can reduce friction as the foot slides distally through the cartridge by centering the pusher. The fingers can also improve dose volume consistency by centering the pusher with respect to the stoppers. The pusher can abut the first stopper when the fluid delivery device is in a resting (e.g., extended) configuration. The active agent can include lyophilized teduglutide.

The cartridge holder can include a radial snap sized and positioned to engage a snap groove in the housing when the cartridge holder is in an extended configuration. Engaging the radial snap with the snap groove can prevent the cartridge holder from rotating inadvertently, for example without the user twisting the device. The radial snap can be sized and positioned to engage a first radial snap aperture and a second radial snap aperture on the housing. Engaging the radial snap with either the first radial snap aperture or the second radial snap aperture radial snap aperture can provide audible and/or tactile feedback of the position of the cartridge holder with respect to the housing (e.g., mixing, priming, etc.).

The cartridge holder can include a radial snap sized and positioned to engage a first radial snap aperture and a second radial snap aperture on the housing. The cartridge holder can be rotatable with respect to the housing from a first position wherein the radial snap engages the first radial snap aperture to a second position wherein the radial snap engages the second radial snap aperture.

A distal end of the cartridge holder can include an internal bevel.

The present disclosure provides a system. The system can include a housing having an internal thread and a button rod groove. The system can include a cartridge holder mechanically coupled to the housing and including an external thread extending at least partially along a length of the cartridge holder and sized to engage the internal thread. The system can include a cartridge disposed within a cartridge holder internal cavity. The system can include a first button rod. The first button rod can include a first drive button and a first wing. A first length of the first button rod between the first wing and a distal end of the first button rod (e.g., a first dosage length) can correspond to a first volume of fluid expelled from the cartridge. The system can include a second button rod. The second button rod can include a second drive button a second wing. A second length of the second button rod between the second wing and a distal end of the second button rod (e.g., a second dosage length) can correspond to a second volume of fluid expelled from the cartridge. The first length can be shorter than the second length, and the first volume can be less than the second volume.

The system can include a foot attachable to a distal end of the first button rod or the second button rod, and the foot can include a pusher to push stoppers within the cartridge. The foot can include a plurality of flexible fingers radially arranged around a common axis extending along the length of the foot. The foot can include a foot groove sized to engage a foot snap of the first button rod or the second button rod.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIGS. 1A and 1B depict a fluid delivery device, according to the present disclosure.

FIGS. 2A-2D are side cross-sectional views of a fluid delivery device, according to the present disclosure.

FIGS. 3A and 3B depict an example cartridge holder, according to the present disclosure.

FIGS. 4A-4E depict an example housing, according to the present disclosure.

FIGS. 5A-5C depict an example button rod, according to the present disclosure.

FIGS. 6A-6C depict an example foot, according to the present disclosure.

FIGS. 7A-7F depict the process of moving fluid delivery device from a reconstitution position to a priming (or retracted) position.

### DETAILED DESCRIPTION

The herein disclosed solution is directed to drug reconstitution devices having a plurality of components movable with respect to one another such that an active agent and a solvent can be reconstituted within the device prior to injection. The plurality of components can provide mechanical, audible, and/or tactile feedback to ensure that the user of the device is properly reconstituting the drug, priming the device for delivery, and injecting the mixed agent. Feedback, safety, and redundancy features are important for drug reconstitution and delivery device for a number of reasons. For one, the drug manufacturer can provide the exact amount of active agent-to-solvent ratio needed for the reconstituted therapeutic. If errors occur during the mixing, this can cause an overdosing or underdosing of the active agent.

Additionally, it is common for the person administering the drug with the delivery device to be someone other than a healthcare provider or pharmacist. These users rely on the device to ensure the mixing is correct and that, once mixed, the accurate amount of drug is delivered. Still further, these devices may be in the hands of people that have dexterity issues, meaning the likelihood of dropping the device may be high. For these reasons, durability and rigidity can be as much of a safety feature as any other feature of the device.

The devices, systems, and methods described herein provide solutions to these problems by providing a fluid delivery system that ensures accurate reconstitution, priming, and delivery. Various devices and methods are disclosed for providing fluid delivery devices, and examples of the devices and methods will now be described with reference to the accompanying figures.

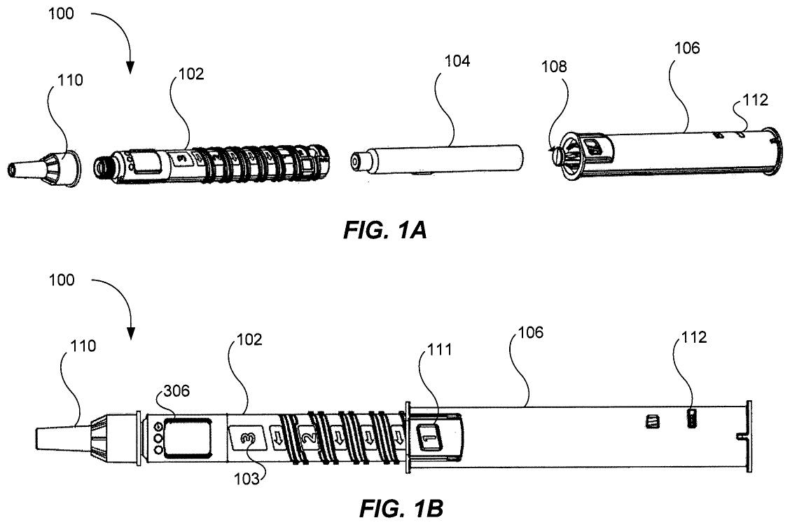

FIG. 1A is a side projection view of a fluid delivery device 100. As described above, the fluid delivery device 100 can include a plurality of components that engage with one another to reconstitute and deliver the fluid (e.g., active agent, therapeutic, drug, or other liquid). For example, the fluid delivery device 100 can be configured to reconstitute and deliver teduglutide, which is a 33-amino acid analog of GLP-2 (glucagon-like peptide 2). The fluid delivery device 100 can include a cartridge holder 102, a cartridge 104, and a housing 106. The cartridge 104 can be pre-loaded with a lyophilized active agent and the solvent that is used to reconstitute the dried active agent, as will be described in greater detail below. The cartridge 104 can be placed within the cartridge holder 102. The cartridge holder 102 can then be mechanically coupled with the housing 106, for example, via a thread on the cartridge holder 102 that corresponds to a thread on the internal surface of the housing 106. The cartridge holder 102 can retract, or move axially, with respect to the housing 106 to reconstitute the drug and to prime the device for delivery.

The fluid delivery device 100 can also include a fixed or modular needle cap 110 that includes a needle for delivering the reconstituted liquid to the patient. The needle cap 110 can include internal threads that engage with distal threads 116 on the cartridge holder 102. The fluid delivery device 100 can include a foot 108 to push stoppers within the cartridge to both reconstitute the active agent and to deliver the active agent through the needle cap 110.

FIG. 1B is a side view of a fluid delivery device 100. As described above, the cartridge holder 102 can be mechanically coupled to the housing 106 via a thread on the cartridge holder 102 that corresponds to a thread on the internal surface of the housing 106. Accordingly, the cartridge holder 102 can retract proximally into the housing 106 by rotating the cartridge holder 102 with respect to the housing 106. The position of the cartridge holder 102 with respect to the housing 106 can indicate to the user the stage of reconstitution or priming of the fluid delivery device 100. For example, a cartridge holder 102 can include one or more position indicators 103 showing the stage the cartridge holder 102 is in with respect to the housing 106. An indicator window 111 in the housing can highlight the position indicators 103. How the axial position of the cartridge holder 102 affects the status (e.g., mixed, primed, etc.) of the fluid delivery device 100 is described in greater detail below.

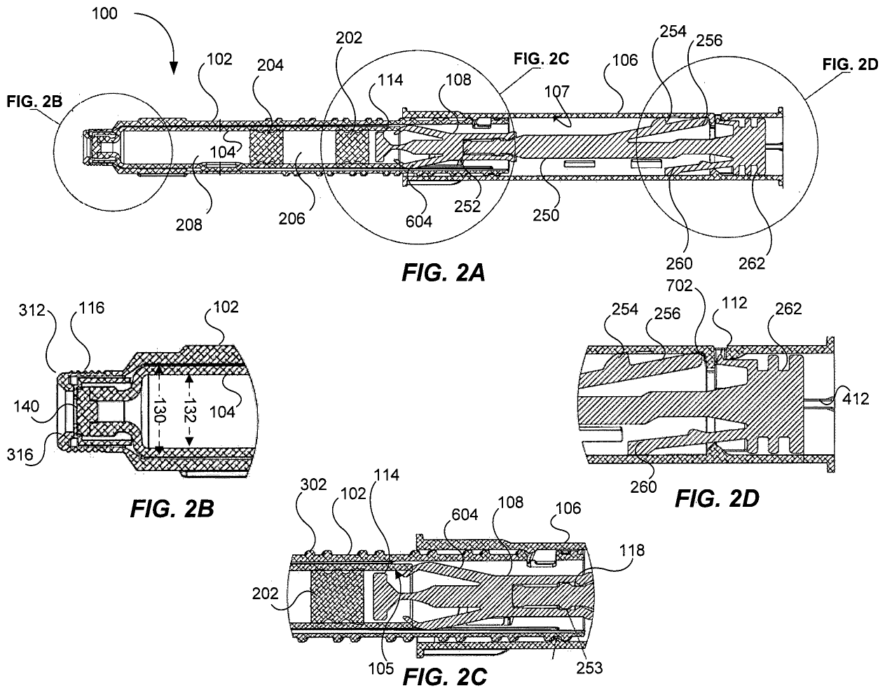

FIGS. 2A-2D are side cross-sectional views of a fluid delivery device 100 taking along a center longitudinal axis of FIG. 1B. FIG. 2A is a cross-sectional view of the fluid delivery device 100, showing an example of how the cartridge holder 102, the cartridge 104, and the housing 106 can engage with each other. The fluid delivery device 100 can include a button rod 250 which can serve as the driving unit to both reconstitute and deliver the active agent. The button rod 250 can include a demounting stop 254, a holding snapper 258, a bypass snapper 260, and a drive button 262, which will all be described in greater detail below. Summarily, these features can be used to prime the fluid delivery device 100 for injection and deliver the therapeutic once primed.

As described above, the cartridge 104 can include stoppers to separate the liquid (e.g., solvent) from the powder (e.g., lyophilized active agent). The cartridge 104 can include a first stopper 202 positioned proximal within an internal cavity 132 of the cartridge 104 and a second stopper 204 positioned distal to the first stopper 202. A first reservoir 206 can be positioned between the first stopper 202 and the second stopper 204. The first reservoir 206 can store the solvent used for reconstitution. A second reservoir 208 can be positioned between the second stopper 204 and a distal tip 140 of the cartridge 104. The second reservoir 208 can store the lyophilized active agent. The second stopper 204 can include a plurality of holes or apertures that enable the solvent to leach into the second reservoir 208 when the first stopper 202 is slid axially toward the second stopper 204.

Referring to FIG. 2B, which is a close-up of a corresponding distal section shown in FIG. 2A, when the cartridge 104 is positioned within an internal cavity 130 of the cartridge holder 102, the distal tip 140 of the cartridge 104 can abut a distal end 312 of the cartridge holder 102. The distal end 312 of the cartridge holder 102 can include a bevel 316 that provides a slight slope to the distal end 312 of the cartridge holder 102. This bevel 316 can eliminate sharp edges at the distal end 312 of the cartridge holder 102 to enable easy cleaning with an alcohol swab. The needle cap 110 can connect to the device at the distal end 312 of the cartridge holder 102 via distal threads 116.

Referring to FIGS. 2A and 2C, the fluid delivery device 100 can include a foot 108 coupled to a distal end 252 of the button rod 250. FIG. 2C in particular is a close-up of a corresponding central section shown in FIG. 2A. The foot 108 can include a pusher 114 that is placed distal to the button rod 250 so as to push the stoppers (e.g., stoppers 202 and 204) through the internal cavity 132 of the cartridge 104. When the button rod 250 is depressed axially, the pusher 114 can exert an axial force onto the first stopper 202 to slide the first stopper 202 axially. Although FIGS. 2A and 2C depict a slight gap between the pusher 114 and the first stopper 202, in some examples it is contemplated that the pusher 114 abuts the first stopper 202 when in a static configuration prior to reconstitution. It has been shown during, developmental testing that positioning the pusher 114 to abut the first stopper 202 can decrease the inadvertent movement of the first stopper 202 axially when subjected to drop testing protocols.

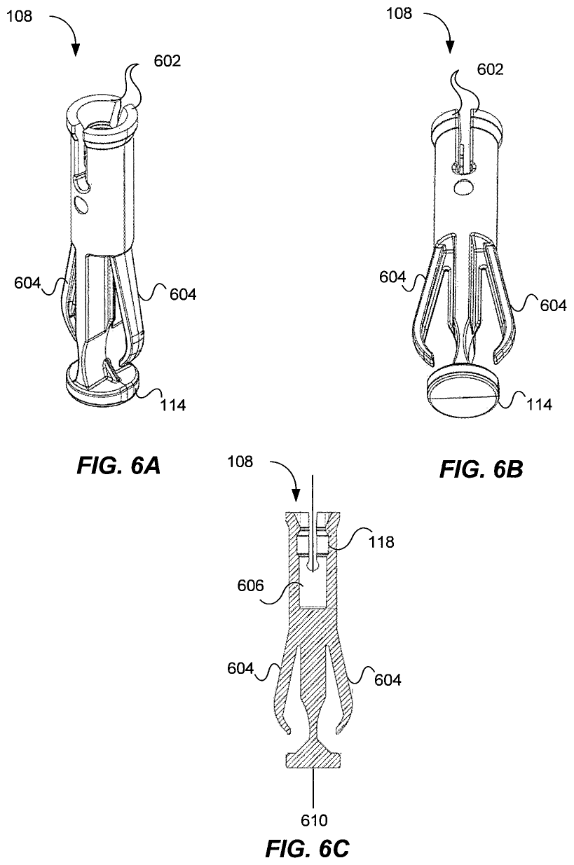

The foot 108 can include a plurality of flexible fingers 604 extending radially from a longitudinal axis 610 of the foot 108. The flexible fingers 604 can extend to exert a radial force on the interior surfaces of the cartridge holder 102 and the cartridge 104. In this manner, the flexible fingers 604 can ensure central positioning of the pusher 114 with respect to the first stopper 202 as the pusher 114 slides axially though the cartridge 104. The foot 108 can include a pair of flexible fingers 604 (as shown in FIGS. 6A-6C) branch proximally and extend distally along the length of the foot 108. The distal end of each of the flexible fingers 604 can be separated from the body of the foot 108 to enable them to flex inwardly toward the axis 610 of the foot 108. The foot 108 can include two flexible fingers 604 extending from opposite sides of the foot 108. It is also contemplated that the foot 108 can include three, four, or more flexible fingers 604. In the case that the foot 108 includes more than two flexible fingers 604, the fingers can be separated equidistant from each other around the axis 610 of the foot 108.

In some examples, the foot 108 can be a separate feature from the button rod 250. For example, the foot 108 can be detachably attachable to the distal end 252 of button rod 250. The foot 108 can include foot groove 118 sized to engage a foot snap 253 on the distal end 252 of the button rod 250. The foot snap 253, for example, can be a flange or protrusion on the distal end 252 of the button rod 250, and the foot groove 118 can be an indentation on the foot 108. In this regard, the foot 108 can attachable engage via the foot snap 253 being slide into and aligned with the corresponding groove 118. However, it is contemplated that the foot 108 can be attached to distal end 252 in other approaches, as needed or required. A particular benefit of separating the foot 108 from the button rod 250, as shown and described, includes enabling the two components to rotate and/or flex independently from each other.

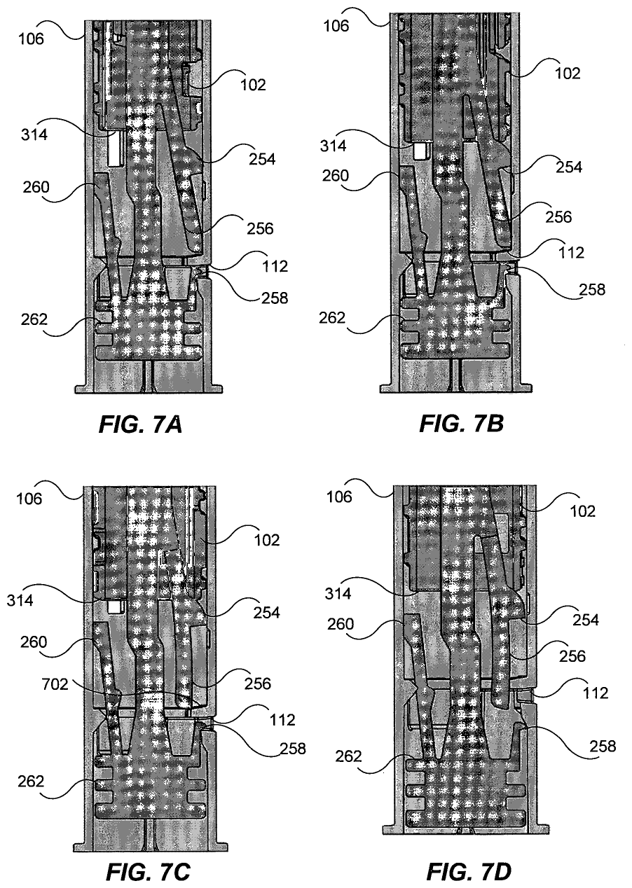

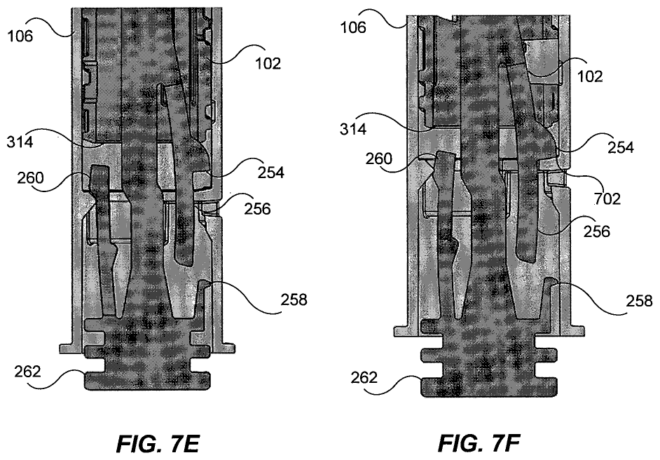

The cartridge holder 102 can have an extended configuration and a retracted configuration with respect to the housing 106. FIG. 2A depicts the extended configuration of holder 102. In this configuration, the cartridge holder 102 has not been rotated so as to move the cartridge holder 102 axially with respect to the housing 106. In the extended configuration, each flexible finger 604 of the foot 108 can engage a proximal end of the cartridge 104. As the cartridge holder 102 is rotated, the cartridge holder 102 moves axially proximally into the housing 106 (e.g., to the right in FIG. 2A), and the pusher 114 moves axially into the cartridge 104, thereby pushing upon the first stopper 202 and the second stopper 204. In the retracted configuration, each flexible finger 604 of the foot 108 can engage an internal surface 105 of the cartridge 104. Friction from the stoppers 202, 204 can cause the button rod 250 to move axially proximally (e.g., to the right in FIG. 2A) and extend from the proximal end 416 of the housing 106, as shown in FIG. 7F.

Referring to FIG. 2D, the cutout view of the button rod 250 within the housing 106 depicts a static state of the button rod 250 when the cartridge holder 102 is in the extended configuration. The holding snapper 258 on the button rod 250 can rest within or engage a holding snapper aperture 112. The holding snapper 258 can retain the button rod 250 after the user injects a therapeutic using the fluid delivery device 100. After the cartridge holder 102 is rotated into its retracted configuration, a proximal end 314 of the cartridge holder 102 can abut the demounting stop 254, causing the retaining hook 256 to deflect inwardly from a housing stop 702 on the housing 106. Once the retaining hook 256 is deflected inwardly, the holding snapper 258 can disengage from the snapper aperture 112, and a drive button 262 of the button rod 250 can slide axially proximally (e.g., to the right in FIG. 2D) to enable the drive button 262 to extend from the housing 106 and provide a surface for a user to press onto the button rod 250 to inject the therapeutic.

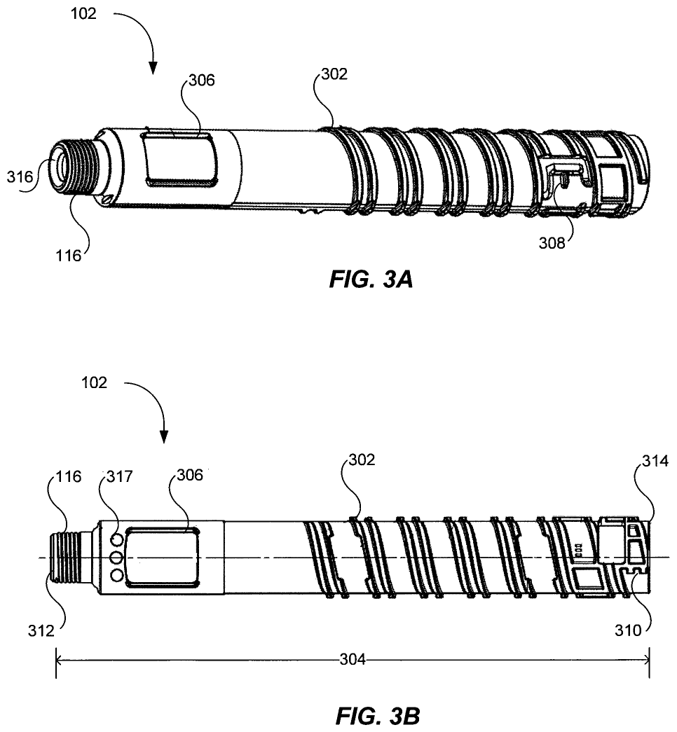

FIGS. 3A and 3B depict an example cartridge holder 102, according to the present disclosure. The cartridge holder 102 can include an external thread 302 extending at least partially along a length 304 of the cartridge holder 102. The external threads 302 can engage with internal threads 404 of the housing 106. At least a portion of the external thread 302 can wrap 360° around the cartridge holder 102. For example, in FIGS. 3A and 3B, the external threads 302 are continuous around the cartridge holder 102, for a total of at least 1440°. However, nothing requires the external thread to be a continuous and uninterrupted path around the cartridge holder 102. A single wrap of 360° can be sufficient to engage with the internal threads 404 of the housing 106. Alternatively, multiple sections of external threads 302 wrapping 360° around the cartridge holder 102 can be placed along the length 304 of the cartridge holder 102, each wrap of threads being separated from each other by a certain distance.

The cartridge holder 102 can include a dosage window 306 placed proximate the distal end 312 of the cartridge holder 102. The dosage window 306 can provide a view of the cartridge 104 placed within the cartridge holder 102. The dosage window 306 can be positioned such that the first reservoir 206 and/or second reservoir 208 are visible during reconstitution of the therapeutic. The cartridge holder 102 can also include dosage indicators 317, which can be dots or indentions that illustrate to the user the total dosage volume of the particular injector. For example, a cartridge holder 102 with three dosage indicators 317 (as shown) can correspond to a 0.3 mL dose, and a cartridge holder 102 with four dosage indicators 317 can correspond to a 0.4 mL dose.

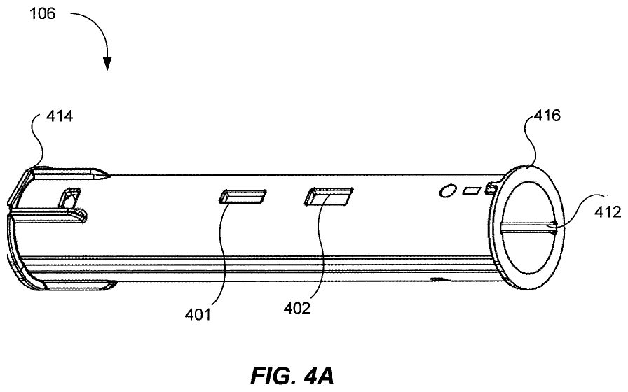

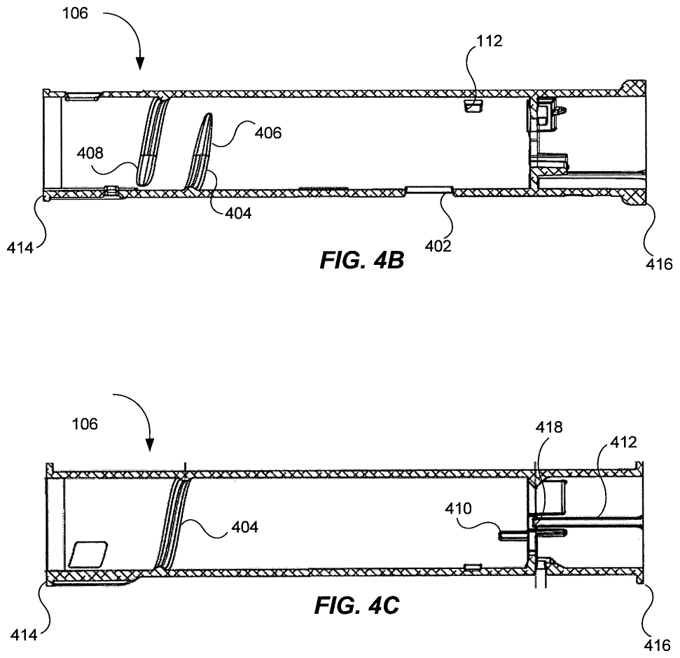

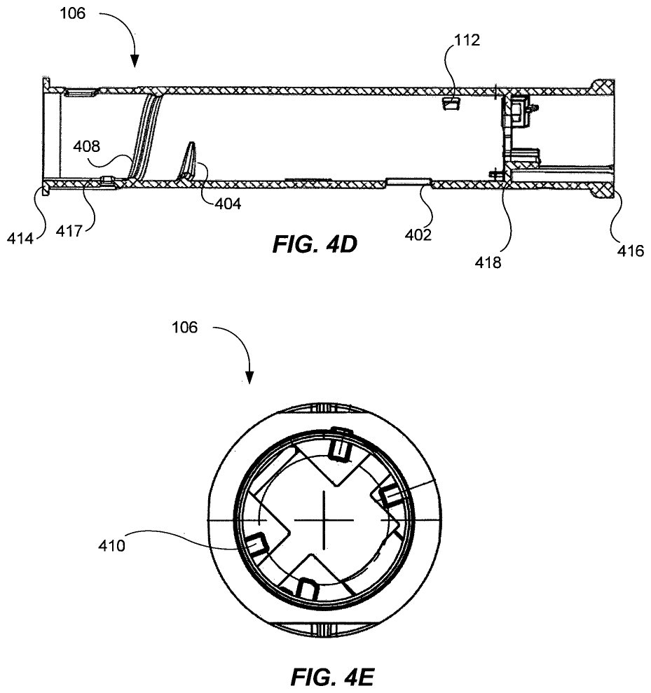

The cartridge holder 102 can include a radial snap 308 sized and positioned to engage a snap groove 417 in the housing 106 when the cartridge holder 102 is in an extended configuration. The snap groove 417 can be seen in FIG. 4D, extending at least partially between the distal end 414 of the housing 106 and the internal threads 404. Engaging the radial snap 308 with the snap groove 417 can prevent the cartridge holder from rotating inadvertently, for example without the user twisting the device. This can be beneficial during shipment of the loaded device, for example. The radial snap 308 can be sized and positioned to engage a first radial snap aperture 401 and/or a second radial snap aperture 402 on the housing 106. The radial snap 308 can provide tactile and/or audible feedback of the different stages of (a) therapeutic reconstitution and (b) priming for injection. The tactile feedback can include a noticeable shake or snap of the fluid delivery device 100 when the radial snap 308 engages the first radial snap aperture 401 and/or the second radial snap aperture 402; the audible feedback can include a noticeable snapping sound when the radial snap 308 engages the first radial snap aperture 401 and/or the second radial snap aperture 402. To illustrate, when the fluid delivery device 100 is at an initial state, the cartridge holder 102 can extend distally from the housing 106. The cartridge holder 102 can be rotated a certain number of turns (e.g., four turns) during the mixing/reconstitution stage. When the cartridge holder 102 reaches the mixing/reconstitution stage, the radial snap 308 can engage with a first radial snap aperture 401 (see, e.g., FIG. 4A), providing tactile and/or audible feedback to the user that the active agent and the solvent have been combined, and the user can then shake the fluid delivery device 100 to mix the therapeutic.

Once mixed, the user can continue rotating the cartridge holder 102 with respect to the housing 106 a certain number of turns (e.g., two turns) to prime the fluid delivery device 100 for injection. Once the device is in the fully-primed configuration, the radial snap 308 can engage with a second radial snap aperture 402 (see, e.g., FIG. 4A), providing tactile and/or audible feedback to the user that the fluid delivery device 100 is primed and ready for injection. The radial snap 308 can be a flange extending from the cartridge holder 102. The first radial snap aperture 401 and/or the second radial snap aperture 402 can be an aperture, hole, or indention in the housing 106. The cartridge holder 102 can include a radial stop 310 that can abut a flange on the housing 106 when the fluid delivery device 100 is fully primed, thereby preventing the cartridge holder 102 from being over-torqued once the device is fully primed.

FIGS. 4A-4E depict an example housing 106, according to the present disclosure. FIG. 4A is a side perspective view of the housing 106; FIGS. 4B-4D are side cross sectional views of the housing 106; and FIG. 4E is an end view of the housing 106. Referring to FIGS. 4B-4D, the housing 106 can include internal threads 404 positioned near a distal end 414 of the housing 106. The internal thread 404 can wrap at least 360° around an interior surface 107 of the housing 106 (as defined above with reference to the external threads 302 of the cartridge holder 102). By wrapping the internal thread 404 at least 360° around the interior surface 107 of the housing 106, a substantial degree of contact (e.g., overlap) can be made between the external threads 302 of the cartridge holder 102, providing a more robust device. It is also contemplated that the internal threads 404 can wrap more than 360° around the interior surface 107 of the housing 106. FIG. 4D depicts an example wherein the internal threads wrap 420° around the interior surface 107 of the housing 106. A greater degree of wrapping of the internal threads 404 can provide additional strength to the connection between the cartridge holder 102 and the housing 106. However, nothing requires the internal thread to wrap 360° around the interior surface 107 of the housing 106, and a lesser degree of wrapping is considered herein. The internal threads 404 can include a beveled first end 406 and a beveled section end 408, which can provide a smooth ramp-like edge to the internal threads 404 to decrease the friction between the internal threads 404 and the external threads 302.

The housing 106 can include an axial stop 410 positioned proximate the proximal end 416 of the housing 106. The axial stop 410 can be sized and positioned to engage a proximal end 314 of the cartridge holder 102 when the cartridge holder 102 is in its fully retracted (primed) position. For example, similar to the radial stop 310 described above on the cartridge holder 102, the axial stop 410 can prevent the cartridge holder 102 from retracting beyond a predetermined position. Preventing the cartridge holder 102 from over-retracting into the housing 106 can reduce underdosing. FIG. 4E depicts an end plan view of the example housing 106. The housing 106 can include more than one axial stops 410 that prevent the button rod 250 from advancing beyond the axial stops 410.

The housing 106 can include button rod groove 412 positioned proximate the proximal end 416 of the housing 106. The button rod groove 412 can be sized to engage the one or more wings 264 of the button rod 250 to enable the button rod 250 to slide both distally and proximally with respect to the housing 106, while also preventing the button rod 250 from rotating with respect to the housing 106. The button rod groove 412 can have a length that defines the total distance the button rod 250 can travel distally within the housing 106. The button rod groove 412 can be cut into the interior surface 107 of the housing 106 and can accept the one or more wings 264 of the button rod 250. A groove stop 418 can be positioned at the distal end of the button rod groove 412 to confine the wings 264 and prevent the button rod 250 from moving axially beyond the groove stop 418.

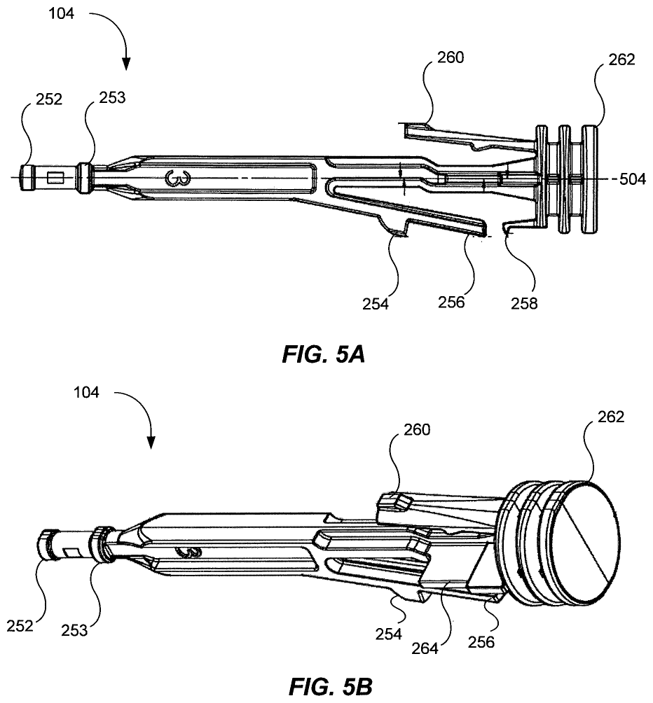

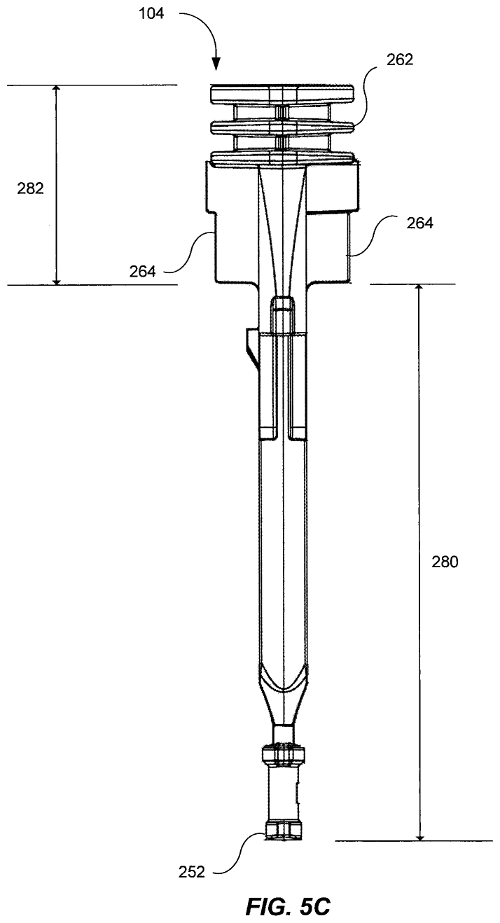

FIGS. 5A-5C depict an example button rod 250, according to the present disclosure. FIG. 5A is a side view of the example button rod 250; FIG. 5B is a side perspective view of the button rod 250; and FIG. 5C is a side view of the button rod 250. The button rod 250 can include a retaining hook 256, a demounting stop 254, a holding snapper 258, a bypass snapper 260, and a drive button 262. The retaining hook 256 can extend from the button rod 250 at a non-zero angle from a longitudinal axis 504 of the button rod 250. The retaining hook 256 can be used to prevent the button rod 250 from inadvertently moving axially with respect to the housing 106 until the cartridge holder 102 comes in contact with the demounting stop 254. As the cartridge holder 102 moves axially with respect to the housing 106, the proximal end 314 of the cartridge holder 102 can contact the demounting step 254 so as to deflect the retaining hook 256 inwardly. The demounting stop 254 can be positioned along a length of the retaining hook 256 and have a smooth, ramped side and a flat side. The smooth side can be positioned distally to enable the proximal end 314 of the cartridge holder 102 to slide upon the demounting stop 254 to disengage the retaining hook 256 from the housing stop 702; and the flat side can be positioned proximate an rest upon the housing stop 702 until the retaining hook 256 is deflected inwardly.

Referring to FIG. 5C, the shape of the button rod 250 can indicate the amount of fluid to be expelled from the cartridge 104. A dosage length 280 of the button rod 250 between the one or more wings 264 and the distal end 252 of the button rod 250 can correspond to a volume of fluid expelled from the fluid delivery device 100. As described above, the wings 264 of the button rod 250 can be sized to slide axially within the button rod groove 412. The button rod 250 can be prevented from moving axially distally with respect to the housing 106 via the groove stop 418 described above. To this end, the dosage length 280 of the button rod 250 between the one or more wings 264 and the distal end 252 of the button rod 250 can define the distance the button rod 250 extends into the cartridge 104. This dosage length 280 can, therefore, be altered based on the amount of fluid intended to be delivered from the cartridge 104. For example, a longer dosage length 280 is equivalent to the button rod 250 (and/or foot 108) extending farther into the cartridge 104 and therefore expelling a greater volume of liquid; a shorter dosage length 280 is equivalent to the button rod 250 (and/or foot 108) extending a shorter distance into the cartridge 104 and therefore expelling a smaller volume of liquid. This alteration of different dosage lengths 280 can be accomplished by inserting a different button rod 250 with a particular dosage length 280 or by adjusting the dosage length 280 of the button rod 250 (e.g., via a button rod 250 that telescopes in length).

Providing a plurality of different button rods 250 with varying dosage lengths 280 enables a manufacturer to vary dosages expelled from a fluid delivery device 100 solely by varying the button rod 250 used within the device. For example, the cartridges holders 102, housings 106, foots 108 can all be constant and the same regardless of the dosage for the particular patient. Further, all cartridges 104 can be manufactured with the same volume of solvent and active agent. Depending on the dosage requirement of the patient, however, the button rod 250 can be adjusted to expel more fluid (i.e., a longer dosage length 280) or less fluid (i.e., a shorter dosage length 280).

After injection, any unused fluid can remain in the distal end of the cartridge 104 (i.e., within the second reservoir 208), and the unused fluid (e.g., the entire cartridge 104) can be discarded. Referring to the dosage window 306 on the cartridge holder 102 described above, once the fluid is expelled, the first stopper 202 and second stopper 204 of the cartridge 104 can be viewable within the dosage window 306, and any remaining fluid can be outside of the view of the dosage window 306. This design can prevent patients from attempting to reuse the remaining fluid in the cartridge, since the remaining fluid is obscured from view by the housing 106.

An example use case for varying dosages can include a fluid delivery device for delivering teduglutide, which is a 33-amino acid analog of GLP-2 (glucagon-like peptide 2). Common volume dosages of reconstituted teduglutide include 0.4 mL and 0.3 mL dosages. All cartridges 104 can be pre-filled with enough active agent (teduglutide) and solvent so as to provide a 0.4 mL dose. For patients that only require the 0.3 mL dose, those patients can receive a device having a button rod 250 that has a shorter dosage length 280 (i.e., one designed for 0.3 mL dosages), thereby allowing approximately 0.1 mL of fluid to remain in the cartridge 104 after injection. As used herein, the terms “about” or “approximately” for any numerical values or ranges indicate a suitable dimensional tolerance that allows the part or collection of components to function for its intended purpose. More specifically, “about” or “approximately” may refer to the range of values ±20% of the recited value, e.g. “about 0.1 mL” may refer to the range of values from 0.0801 mL to 0.1199 mL.

Alternatively or additionally to choosing different button rods 250 based on their dosage length 280, different length foots 108 can be selected for different dosage volumes. As described above, the foot 108 can be attached to the distal end 252 of the button rod 250. To this end, lengthening the foot 108 can also increase the overall length of the device that extends into the cartridge 104, and shortening the foot 108 can also decrease the overall length of the device that extends into the cartridge 104. Further, the foot groove 118 within the foot 108 (e.g., where the distal end 252 of the button rod 250 can attach to the foot 108) can be positioned to alter the overall length of the button rod 250/foot 108 combined device. Selection of the particular button rod 250 and/or foot 108 for a particular dosage volume can be made by a manufacturer, so that the end user only receives the fluid delivery device 100 that is customized for their dosing regimen.

Referring again to FIG. 5C, to ensure that the overall length of all button rods 250 remain the same even for systems that include button rods 250 with varying dosage lengths 280, the button rods 250 can have a button length 282 defined as a length between the distal end of the wings 264 (e.g., bottom in FIG. 5C) to the proximal end of the drive button 262. For button rods 250 that have a shorter dosage length 280, the component can include a longer button length 282; for button rods 250 that have a longer dosage length 280, the component can include a shorter button length 282.

FIGS. 6A-6C depict an example foot 108, according to the present disclosure. FIGS. 6A and 6B are perspective views; and FIG. 6C is a side cross sectional view of the foot 108. The foot 108 can include a plurality of button rod snaps 602 that define an open, upper, proximal end of the foot and are separated by grooves extending parallel to the longitudinal axis 610 of the foot 108. The button rod snaps 602 can enable the distal end 252 of a button rod 250 to be inserted within a cavity 606 in the foot 108, while preventing the button rod 250 from being pulled axially from the foot 108. A foot groove 118 can be disposed within the cavity 606. As described above, the foot groove 118 can be sized and positioned to engage a foot snap 253 on the distal end 252 of the button rod 250. The distal end 252 of the button rod 250 can rotate within the cavity 606 of the foot 108. Separating the foot 108 from the button rod 250 can reduce the friction subjected to the stoppers 202, 204 as the cartridge holder 102 (and cartridge 104) are rotated for reconstitution and priming. For example, as the cartridge holder 102 (and thus cartridge 104) is rotated from its extended configuration, to its reconstitution/mixing position, and to is fully primed (retracted) position, the pusher 114 of the foot 108 can abut the first stopper 202 and rotate along with the cartridge 104. The button rod 250, however, does not rotate, so the foot 108 can rotate around the distal end 252 of the button rod 250. A lubricant (e.g., a silicone lubricant) can be added within the cavity 606 so as to further decrease the friction of the foot/button rod connection.

FIGS. 7A-7F depict the process of moving fluid delivery device 100 from a reconstitution position to a primed (or retracted) position. At FIG. 7A, the cartridge holder 102 has been rotated such that the solvent and active agent in the cartridge 104 is mixed. The holding snapper 258 rests within the holding snapper aperture 112, holding the button rod 250 in its resting position.

As shown in FIG. 7B, additional rotations of the cartridge holder 102 can cause the cartridge holder 102 to continue to move axially proximally (e.g., down in FIGS. 7A-7F) until a proximal end 314 of the cartridge holder 102 abuts the demounting stop 254. In FIG. 7C, the cartridge holder 102 is rotated until the proximal end 314 slides along the demounting stop 254, causing the retaining hook 256 to deflect inwardly such that the retaining hook 256 is no longer abutting the housing stop 702. In FIG. 7D, the cartridge holder 102 continues to move axially proximally. The friction of the stoppers 202, 204 pushing against the foot 108 (and, therefore, button rod 250) causes the entire button rod 250 to move axially proximally.

In FIG. 7E, the cartridge holder 102 continues to move axially proximally. The bypass snapper 260 can contact a housing ramp 704. In FIG. 7F, the bypass snapper 260 is deflected inwardly by the housing ramp 704, and the demounting stop 254 abuts the housing stop 702. The flat surface of the demounting stop 254 abutting the housing stop 702 can stop the button rod 250 from moving any farther axially. As described above, axial stops 410 (not shown in FIG. 7E), can also stop the axial movement of the cartridge holder 102. At this point, the device is primed for injection, and the drive button 262 can extend from the proximal end 416 of the housing 106, thereby providing the user a surface in which press upon to inject the therapeutic. Once depressed, the bypass snapper 260 can slide across the housing ramp 704, providing additional axial force as the button rod 150 slides distally to press upon the stoppers 202, 204.

As described above, the friction of the stoppers 202, 204 within the cartridge 104 can cause the button rod 250 to move axially, as shown in FIGS. 7A-7B. The fluid delivery device 100 also provides a redundancy to ensure button rod 250 slides axially with respect to the housing 106, in the case the friction of the stoppers 202, 204 is insufficient. The proximal end 314 of the cartridge holder 102 can abut the bypass snapper 260, and the cartridge holder 102 can push the bypass snapper 260 axially proximally with respect to the housing 106.

Examples of the present disclosure can also be implemented according to at least the following clauses:

Clause 1: A method of using the fluid delivery device 100 described above, the method comprising: rotating the cartridge holder 102 with respect to the housing 106 causing the cartridge holder 102 to move axially proximally through the housing 106 from a first position wherein a radial snap 308 on the cartridge holder 102 engages a first radial snap aperture 401 on the housing 106 to a second position wherein the radial snap 308 engages a second radial snap aperture 402 on the housing 106; and rotating the cartridge holder 102 with respect to the housing 106 causes the button rod 250 to move axially distally through the cartridge 104.

Clause 2: The method of clause 1, further comprising: pushing a first stopper 202 distally through the cartridge 104 via a foot 108 coupled to a distal end 252 of the button rod 250 such that the first stopper 202 moves axially distally toward a second stopper 204 within the cartridge 104.

Clause 3: The method of clause 2, wherein the foot 108 is detachably attachable to the distal end 252 of the button rod 250.

Clause 4: The method of clause 2, wherein moving the first stopper 202 axially distally toward the second stopper 204 causes an active agent disposed between a distal tip 140 of the cartridge 104 and the second stopper 204 to mix with a solvent disposed between the second stopper 204 and the first stopper 202, thereby creating a reconstituted liquid.

Clause 5: The method of clause 4, further comprising pressing the drive button 262 axially distally such that the first stopper 202 and the second stopper 204 move axially distally and expel the reconstituted liquid from a distal tip 140 of the cartridge 104.

Clause 6: The method of clause 5, further comprising: expelling only a portion of the reconstituted liquid from the distal tip 140.

Clause 7: The method of clause 6, wherein approximately 0.1 mL of reconstituted liquid remains in the cartridge 104 when the portion of the reconstituted liquid is expelled from the distal tip 140.

Clause 8: The method of clause 4, further comprising engaging the radial snap 308 with the second radial snap aperture 402 thereby causing an audible feedback indicative of the fluid delivery device 100 being primed for injection.

Clause 9: The method of clause 4, wherein the active agent comprises lyophilized teduglutide.

Clause 10: The method of clause 1, further comprising pushing a first stopper 202 distally through the cartridge 104 via a foot 108 coupled to a distal end 252 of the button rod 250 such that the first stopper 202 moves axially distally toward a second stopper 204 within the cartridge 104.

Clause 11: The method of clause 1, further comprising: abutting, with a proximal end 314 of the cartridge holder 102, the demounting stop 254 as the cartridge holder 102 moves axially proximally through the housing 106; and deflecting, with the proximal end 314 of the cartridge holder 102, the retaining hook 256 inwardly such that the retaining hook 256 bypasses a housing stop 702 positioned proximate a proximal end of the housing 106.

Clause 12: The method of clause 11, wherein the step of rotating the cartridge holder 102 with respect to the housing 106 further causes the button rod 250 to move axially with respect to the housing 106 such that the drive button 262 extends from a proximal end 416 of the housing 106.

Clause 13: The method of clause 12, further comprising pushing a first stopper 202 distally through the cartridge 104 via a foot 108 coupled to a distal end 252 of the button rod 250 such that the first stopper 202 moves axially distally toward a second stopper 204 within the cartridge 104, wherein friction of the first stopper 202 sliding within an internal cavity 132 of the cartridge 104 causes the button rod 250 to move axially with respect to the housing 106.

Clause 14: The method of clause 13, pushing, with a proximal end 314 of the cartridge holder 102, the bypass snapper 260 axially proximally with respect to the housing 106.

The descriptions contained herein are examples of embodiments of the disclosure and are not intended in any way to limit the scope of the disclosure. As described herein, the disclosure contemplates many variations and modifications of the aspiration device including using alternative geometries of structural elements, combining shapes and structural elements from various example embodiments, using alternative materials, etc. These modifications would be apparent to those having ordinary skill in the art to which this disclosure relates and are intended to be within the scope of the claims which follow.

1. A fluid delivery device comprising:
a housing comprising an internal thread;
a cartridge holder mechanically coupled to the housing and comprising an external thread extending at least partially along a length of the cartridge holder, at least a portion of the external thread wrapping 360° around the cartridge holder;
a cartridge disposed within a cartridge holder internal cavity; and
a button rod comprising:
a retaining hook;
a demounting stop extending at a non-zero angle from a longitudinal axis of the button rod;
a holding snapper;
a bypass snapper extending at a non-zero angle from the longitudinal axis of the button rod; and
a drive button, and
a foot coupled to a distal end of the button rod and comprising a pusher.

2. The fluid delivery device of claim 1, wherein the housing further comprises a button rod groove, and the button rod further comprises a wing sized to engage the button rod groove.

3. The fluid delivery device of claim 2, wherein a first length of the button rod between the wing and a distal end of the button rod corresponds to a volume of fluid expelled from the fluid delivery device.

4. (canceled)

5. The fluid delivery device of claim 1, wherein the foot comprises a plurality of flexible fingers radially arranged around a common axis extending along the length of the foot, wherein:
the cartridge holder has an extended configuration and a retracted configuration with respect to the housing;
in the extended configuration, each flexible finger of the plurality of flexible fingers extending radially from the foot engages a proximal end of the cartridge; and
in the retracted configuration, each flexible finger of the plurality of flexible fingers extending radially from the foot engages an internal surface of the cartridge.

6. (canceled)

7. The fluid delivery device of claim 5, wherein, when in the retracted configuration, the demounting stop abuts a housing stop positioned proximate a proximal end of the housing.

8. The fluid delivery device of claim 1, wherein the foot further comprises a foot groove sized to engage a foot snap of the button rod.

9. The fluid delivery device of claim 1, wherein the foot is detachably attachable to the distal end of the button rod.

10. The fluid delivery device of claim 1, wherein the cartridge holder is rotatable with respect to the housing, and wherein the retaining hook is inwardly deflectable by a proximal end of the cartridge holder.

11. The fluid delivery device of claim 1, wherein the housing comprises a holding snapper aperture sized to engage the holding snapper.

12. The fluid delivery device of claim 1, wherein the internal thread wraps at least 360° around an interior surface of the housing.

13. The fluid delivery device of claim 12, wherein the internal thread comprises a beveled first end and a beveled second end to reduce friction as the cartridge holder rotates with respect to the housing.

14. The fluid delivery device of claim 1, wherein the internal thread wraps at least 420° around an interior surface of the housing.

15. The fluid delivery device of claim 1, wherein the housing comprises an axial stop sized to engage a proximal end of the cartridge holder, wherein the cartridge holder has an extended configuration and a retracted configuration with respect to the housing, and the axial stop is positioned to inhibit the cartridge holder from sliding proximally beyond a predetermined position thereby reducing underdosing.

16. (canceled)

17. The fluid delivery device of claim 1, wherein the cartridge comprises:
a distal tip;
a first stopper;
a second stopper disposed between the distal tip and the first stopper;
an active agent disposed between the distal tip and the second stopper; and
a solvent disposed between the second stopper and the first stopper,
wherein the foot comprises a plurality of flexible fingers extending radially from the foot and centering the pusher centrally with respect to the first stopper and improving dose volume consistency by centering the foot as the foot slides distally through the cartridge.

18-19. (canceled)

20. The fluid delivery device of claim 17, wherein the active agent comprises lyophilized teduglutide.

21. The fluid delivery device of claim 1, wherein the cartridge holder further comprises a radial snap sized and positioned to engage a snap groove in the housing when the cartridge holder is in an extended configuration.

22. The fluid delivery device of claim 1, wherein the cartridge holder further comprises a radial snap sized and positioned to engage a first radial snap aperture and a second radial snap aperture on the housing, wherein engaging the radial snap with either the first radial snap aperture or the second radial snap aperture provides an audible feedback.

23. The fluid delivery device of claim 1, wherein:
the cartridge holder further comprises a radial snap sized and positioned to engage a first radial snap aperture and a second radial snap aperture on the housing; and
the cartridge holder is rotatable with respect to the housing from a first position wherein the radial snap engages the first radial snap aperture to a second position wherein the radial snap engages the second radial snap aperture.

24. (canceled)

25. A method of using the fluid delivery device of claim 1, the method comprising:
rotating the cartridge holder with respect to the housing causing the cartridge holder to move axially proximally through the housing from a first position wherein a radial snap on the cartridge holder engages a first radial snap aperture on the housing to a second position wherein the radial snap engages a second radial snap aperture on the housing; and
rotating the cartridge holder with respect to the housing causing the button rod to move axially distally through the cartridge, and
pushing a first stopper distally through the cartridge via the foot such that the first stopper moves axially distally toward a second stopper within the cartridge.

26-38. (canceled)

39. A system comprising:
a housing comprising an internal thread and a button rod groove;
a cartridge holder mechanically coupled to the housing and comprising an external thread extending at least partially along a length of the cartridge holder and sized to engage the internal thread;
a cartridge disposed within a cartridge holder internal cavity;
a first button rod comprising:
a first drive button; and
a first wing, a first length of the first button rod between the first wing and a distal end of the first button rod corresponding to a first volume of fluid expelled from the cartridge; and
a second button rod comprising:
a second drive button; and
a second wing, a second length of the second button rod between the second wing and a distal end of the second button rod corresponding to a second volume of fluid expelled from the cartridge,
wherein the first length is shorter than the second length, and
wherein the first volume is less than the second volume, and
a foot attachable to a distal end of the first button rod or the second button rod and comprising a pusher.

40-42. (canceled)
